(19) United States
(12) Patent Application Publication
(10) Pub. No.: US 2006/0172339 A1
(43) Pub. Date: Aug. 3, 2006

(54) Particle-based multiplex assay for identifying glycosylation

(75) Inventors: Wayne F. Patton, Newton, MA (US); Mack J. Schermer, Belmont, MA (US); Mark N. Bobrow, Lexington, MA (US)

Correspondence Address: WILMER CUTLER PICKERING HALE AND DORR LLP, 60 STATE STREET, BOSTON, MA, 02109, US

(73) Assignee: PerkinElmer LAS, Inc., Boston, MA (US)

(21) Appl. No.: 11/289,060

(22) Filed: Nov. 29, 2005

Related U.S. Application Data
(60) Provisional application No. 60/631,394, filed on Nov. 29, 2004.

(51) Int. Cl. G01N 33/53; G06F 19/00

(52) U.S. Cl. 435/7.1; 702/19

(57) ABSTRACT

The present invention provides systems, methods and kits for performing multiplexed assays for glycoproteins using encoded particles as supports for glycoprotein specific binding agents.

### BACKGROUND OF THE INVENTION

This invention is in the field of multiplex assay platforms. In particular, it is in the field of multiplexed assays for determining the glycosylation states of molecules, including proteins and lipids.

In eukaryotes, oligosaccharides are commonly co- or post-translationally attached to molecules, such as proteins and lipids, by a variety of glycosidases and glycosyltransferases. For example, carbohydrate residues are enzymatically or chemically attached to proteins through N-glycosidic linkage via the amide nitrogen of asparagine, through O-glycosidic linkage via the hydroxyl of serine, threonine, hydroxylysine or hydroxyproline or through glycosyl phosphatidylinositol (GPI) anchoring that is directed by a COOH terminus signal sequence subsequently removed during the attachment process. Extracellular matrix and cell surface proteins are particularly rich in glycosylation.

Glycosylation contributes to the proper folding, biological activity, immunogenicity, clearance rate, solubility, stability, and protease and/or lipase resistance of proteins and lipids. Indeed, glycosylation of proteins is critical to the adhesiveness of microorganisms and cells, cellular growth control, cell migration, tissue differentiation, and inflammatory reactions. Alterations in glycosylation profiles of proteins and lipids are often useful indicators for the assessment of disease states. As biomedical investigations have increasingly involved proteomics, there has been renewed interest in methodologies for the rapid and sensitive identification of glycosylated molecules, such as glycoproteins and glycolipids.

The use of multiplex assays for identifying components in complex sample has many applications in the fields of drug discovery, medical research and biological research. For example, microarrays of many types are widely used in these discovery and research fields. RNA expression arrays and antibody arrays on planar supports made of glass, silicon, or on thin gel or membrane layers coated on top of those supports are the most common. Complex biological samples containing unknown mixtures of analytes complementary to the specific binding pair members immobilized on the array are applied and allowed to incubate. The complementary specific binding pair members in the sample solution are thus captured by the immobilized binding pair members. A labeling mechanism is utilized to cause the mated binding pairs to produce a detectable signal, usually an optical signal such as fluorescence or a color change. The labeling can be accomplished by many methods. The simplest ones utilize chemical or enzymatic labeling of all or most potential binding molecules in the sample. More specific results can be obtained at the expense of more complicated assay development by using a “sandwich” assay, wherein a second binding pair member, different from any previously immobilized on the array but with specific affinity to each captured substance at each array location, are labeled and incubated on the array in a second step.

However, microarrays have several well-known problems. First, the creation of the array is complex and requires expensive specialized equipment and particular skills in the people who operate it. Printing arrays with well-controlled nanoliter or picoliter amounts of immobilized capture molecules at each spot, thereby controlling the signal-producing potential of each spot, is challenging. The concentrations of the solutions being printed change with evaporation during the printing process, for example. Also, the local hydrophobicity variations of the array substrate on a micro scale have large effects on the size and hence area concentration of the printed spots, also affecting their signal-producing potentials.

Second, collection of data from an incubated microarray requires imaging the array, wherein the appropriate optical property (e.g. fluorescence, color, etc.) is measured across the array on a pixel-by-pixel basis in the form of an electronic image. The resulting image must be segmented into “spot” and “background” areas and the values of the pixels in a spot segment need to be consolidated into a single value representing that spot's signal. This process is challenging, as the exact size, shape and location of each spot all have substantial tolerances upon them due to limitations to the array printing processes. Some pixels span the boundary between the spot and the background and their values are at an intermediate level between the two levels; these pixels must be discarded. When there is substantial spatial noise in the image, e.g., variation of signal levels from pixel to pixel due to unintended limitation of the microarray preparation and imaging, this segmenting of the image and consolidation into a signal value is particularly challenging. The spot printing process, for example, generally produces spots with substantial concentration variations across the spots when examined on the scale of typical microarray imaging pixels, from about 3 μm to about 30 μm. For these and other reasons microarrays have become well known for reproducibility problems and their use has been largely excluded to date from diagnostics and other critical settings.

It would thus be useful to have a non-microarray method that could simultaneously identify the concentrations or amounts of glycosylation on a multitude of molecules in a complex sample. Further, it would be useful to have a non-microarray method that could simultaneously identify a multitude of structural features possessed by a glycosylated molecule.

### SUMMARY OF THE INVENTION

The invention provides a rapid method for simultaneously identifying glycosylated molecules in a biological sample. Non-limiting examples of glycosylated molecules that can be identified with the present invention include glycoproteins, proteoglycans, oligosaccharides, lipopolysaccharides, glycopeptides, glycosaminoglycans, polysaccharides, glycolipids, gangliosides, glycohormones, cerebrosides and glycosylsphingolipids.

Accordingly, in one aspect, the invention provides a method for simultaneously detecting one or more glycosylated molecules in a number of labeled samples. The method includes contacting the number of labeled samples with a number of aliquots comprising a plurality of particle sets, wherein each particle in a particle set is coated with a glycosylated molecule specific binding agent and wherein each particle in the particle set is encoded with a different identifier, and wherein the number of samples is greater than or equal to the number of aliquots containing the plurality of particle sets; and identifying glycosylated molecules within the sample by collecting identifier data and collecting binding agent interaction data. In some embodiments, the sample is a biological sample.

In a further aspect, the invention provides a method for characterizing a sugar residue on a labeled glycosylated molecule, comprising contacting the labeled glycosylated molecule with a plurality of particle sets, wherein each particle in a particle set is coated with a glycosylated molecule specific binding agent and wherein each particle in the particle set is encoded with a different identifier and collecting identifier data and collecting binding agent interaction data. The labeled glycosylated molecule is next treated with a glycosidase, and then allowed to contact the plurality of particle sets. Identifier data and binding agent data are collected, where the identifier data and the binding agent interaction data characterize one or more sugar residues on the labeled glycosylated molecule.

In certain embodiments, the glycosylated molecule is a glycoprotein, proteoglycan, oligosaccharide, lipopolysaccharide, glycopeptide, glycosaminoglycan, polysaccharide, glycolipid, ganglioside, glycohormone, cerebroside, or glycosylsphingolipid. In some embodiments, the glycosylated molecule specific binding agent is an antibody, a lectin, an aptamer, a protein, or a glycoprotein.

In some embodiments, the identifier data and binding agent interaction data are collected by a reading instrument. In some embodiments, the binding agent interaction data is collected by fluorescence detection. In some embodiments, the identifier is a barcode or is a fluorescent label.

In particular embodiments, the number of particle sets in the plurality of particle sets is between about 2 and about 400. In some embodiments, each particle set comprises between about 1 and about 5,000 particles.

In another aspect, the invention provides a kit for performing simultaneous assays of one or more glycosylated molecules in a sample. The kit includes one or more aliquots comprising a plurality of particle sets, wherein each particle in a particle set is coated with a glycosylated molecule specific binding agent and wherein each particle in the particle set is encoded with a different identifier, and a reagent for attaching a label to the glycosylated molecule.

In another aspect, the invention provides a kit for characterizing a carbohydrate residue on a glycosylated molecule. The kit includes a plurality of particle sets, wherein each particle in a particle set is coated with a glycosylated molecule specific binding agent and wherein each particle in the particle set is encoded with a different identifier; a glycosidase; and a reagent for attaching a label to the glycosylated molecule.

In some embodiments, the number of particle sets in the plurality of particle sets in the kits is between about 2 and about 400. In certain embodiments of the kits, the identifier is a barcode or is a fluorescent label. In some embodiments, the glycosylated molecule specific binding agent is an antibody, a lectin, an aptamer, a protein, or a glycoprotein.

In particular embodiments, the kits include a vessel in which to perform the assay. In some embodiments, the kits include instructions for using the kits to perform the assay.

In a further aspect, the invention provides a system for performing simultaneous assays of one or more glycosylated molecules in a sample. The system includes one or more aliquots comprising a plurality of particle sets, wherein each particle in a particle set is coated with a glycosylated molecule specific binding agent and wherein each particle in the particle set is encoded with a different identifier; a vessel in which to perform the assay; a reagent for attaching a label molecules to the glycosylated molecule; and a reading instrument.

In some embodiments, the number of particle sets in the plurality of particle sets is between about 2 and about 400. In some embodiments, the glycosylated molecule specific binding agent is an antibody, a lectin, an aptamer, a protein, or a glycoprotein.

In certain embodiments, the identifier is a barcode or is a fluorescent label.

### BRIEF DESCRIPTION OF THE DRAWINGS

FIG. 1 is a process flow diagram showing the preparation of and running of a non-limiting example of an encoded particle based multiplexed assay.

FIG. 2 is a diagram of a hologram-encoded multiplex assay particle.

FIG. 3 is a schematic representation of the particle of FIG. 2 coated with an antibody, a non-limiting molecule of a specific glycoprotein binding pair member.

FIG. 4 is a schematic representation of the antibody-coated particle of FIG. 3, where a plurality of the antibodies have bound to their complementary glycoproteins.

### DETAILED DESCRIPTION OF THE PREFERRED EMBODIMENTS

The invention stems from the inventors' discovery that glycosylation status can be determined on numerous analytes simultaneously in a multiplex assay using glyco-reactive identifier-encoded particles. The invention provides an encoded particle system for performing multiplexed assays for glycosylated molecules. The patents and publications identified in this specification are within the knowledge of those skilled in this field and are each hereby incorporated by reference in their entirety.

In one aspect, the invention provides a method for simultaneously detecting one or more glycosylated molecules in a number of samples. The method includes contacting the number of samples with a number of aliquots containing a plurality of particle sets, wherein each particle in a particle set is coated with a glycosylated molecule specific binding agent and wherein each particle in the particle set is encoded with a different identifier, and wherein the number of samples is greater than or equal to the number of aliquots containing the plurality of particle sets. The glycosylated molecules within the sample are identified by collecting identifier data and collecting binding agent interaction data.

As used herein, by “sample” is meant any solid, liquid, or gas suspected of containing a glycosylated molecule. The glycosylated molecule in the sample may have a known glycosylation pattern, or it may have an unknown glycosylation pattern. In some embodiments, the sample is a biological sample, such as a tissue sample, cerebrospinal fluid, blood, lymph fluids, tissue homogenate, interstitial fluid, cell extracts, mucus, saliva, sputum, stool, physiological secretions, or other similar fluids or cells. In some embodiments, the sample is a lysate prepared from cells taken either from a biopsy (in vivo) or grown in tissue culture (in vitro). In some embodiments, the sample is conditioned media from cells grown in tissue culture. Additional non-limiting samples include samples obtained from an environmental source such as sewage, soil, water (e.g., from a pond, lake, or ocean), or air; or from an industrial source such as taken from a waste stream, a water source, a supply line, or a production lot. Industrial sources also include fermentation media, such as from a biological reactor or food fermentation process such as brewing; or foodstuffs, such as meat, grain, produce, eggs, or dairy products.

By “labeled sample” is meant that the sample (or the molecules in the sample) are attached to a label. Any suitable label that is detectable with a reading machine may be used to label a sample. Typically, the label is simply mixed with the molecules, and allowed to react, forming a covalent or non-covalent bond. Non-limiting labels include anthranilic acid (2-aminobenzoic acid; 2-AA), 1-phenyl-3-methyl-5-pyrazolone (PMP), phenylhydrazine (PHN), Fluorescein (FITC), R-Phycoerythrin (PE), Cy5, Cy3, Texas Red, Propidium Iodide (PI), or radiolabels (e.g., 32P, 3H), deuterium, biotin, and streptavidin.

The use of multiplex assays for identifying glycosylated molecules in a sample has many applications in the fields of drug discovery, medical research and biological research. As used herein, by “glycosylated molecule” or simply “glyco-molecule” is meant any molecule that has a carbohydrate (also called saccharide or sugar) residue on it. Thus, the term includes, without limitation, cell surface glycoconjugates, microbial surfaces (e.g., from bacteria), glycoproteins, proteoglycans, oligosaccharides, lipopolysaccharides, glycopeptides, glycosaminoglycans, polysaccharides, glycolipids, gangliosides, glycohormones, cerebrosides and glycosylsphingolipids.

Prior to being assayed in accordance with the invention, the molecules in the sample may be attached to a label. Methods for attaching labels to molecules such as lipids and glycoproteins are well known and may be by a covalent or non-covalent bonds. (For example, the Sigma-Aldrich company (through its Fluka subsidiary in Switzerland) provides a number of fluorescent labels with instruction for attaching them to molecules.) Non-limiting labels include tetramethylrhodamine isothiocyanate, fluorescein isothiocyanate (FITC), Cyanine 3 succinimidyl ester or any of a variety of other labels.

For the multiplex assay of the invention, sets of particles are employed, wherein each set is encoded (i.e., labeled or marked) with a different identifier. As used herein, by the term “identifier” is meant any means of distinguishing one set of particles from another set of particles, or means of distinguishing a particle from one set from one or more particles from another set. Thus, non-limiting identifiers of the invention include encoding particles with optical barcodes, holographic barcodes, particles having different fluorescent intensities or ratios (e.g., one particle set has a green dye:blue dye ratio of 50:50, another has a ratio of 60:40), particles having different colors or size, particles having incorporated into them a radio frequency identification (RFID) mechanism or any other optical, mechanical, or electronic means for distinguishing a particle of one set from a particle of another set. As used herein, by “set” means one or more particle, wherein each particle within a set is identical. Accordingly, all the particles within one set are encoded with the same identifier.

In some embodiments, the identifier is a barcode. The barcode may be optical or holographic.

Methods for engraving particles with a barcode, and devices for reading data from such particles have been described (see, e.g., U.S. Patent Publication Nos. US2004-0179267 (Moon et al., “Method and apparatus for labeling using diffraction grating-based encoded optical identification elements”); US2004-0132205 (Moon et al., “Method and apparatus for aligning microbeads in order to interrogate the same”); US2004-0130786 (Putnam et al., “Method of manufacturing of diffraction grating-based optical identification element”); US2004-0130761 (Moon et al., “Chemical synthesis using diffraction grating-based encoded optical elements”); US2004-0126875 (Putnam et al., “Assay stick”); US2004-0125424 (Moon et al., “Diffraction grating-based encoded micro-particles for multiplexed experiments”); and US2004-0075907 (Moon et al., “Diffraction grating-based encoded micro-particles for multiplexed experiments”). These barcodes are advantageous in that a very large number of particle types can be differentiated and identified using the large number of potential codes and by the environmental robustness of the barcode gratings recorded permanently inside the glass particles. A commercially available system using barcode-encoded particles is the CyVera system available from Illumina, Inc., San Diego, Calif.

In some embodiments, the identifier is a fluorescent label (i.e., a fluorescent dye or a fluorophore), such that particles of different sets have fluorescent labels that have different excitation and/or emission wavelengths. In some embodiments, the identifier is a ratio of fluorescence, such that particles of different sets have different ratios of fluorescence (e.g., a blue dye:green dye ratio in one set of 50:50 and a blue dye:green dye ratio in another set of 60:40). Fluorescently labeled particles have been described. For example, U.S. Pat. No. 5,981,180 (Chandler et al., “Multiplexed analysis of clinical specimens apparatus and methods”) describes a particle-based multiplex assay system in which the particles are encoded by mixtures of various proportions of two or more fluorescent dyes impregnated into polymer particles. The assay signal is reported by a fluorescent label that has excitation and emission wavelengths substantially separated from the particle-identification dyes. The particles are read by a flow-cytometer type of instrument that draws particle from an assay vessel, such as a microplate well, and interrogates each particle optically for its particle identity and its assay signal as it passes through a reading capillary. This system has been implemented commercially as the Luminex Corp. (Austin, Tex.) xMAP product line and is used for a variety of assay types in research, drug discovery and in some FDA-approved diagnostic applications. In addition, U.S. Pat. No. 5,028,545 (Soini, “Biospecific multianalyte assay method”) describes a similar particle-based multiplexed assay system, but where time-resolved fluorescent is utilized rather than the prompt fluorescence described by U.S. Pat. No. 5,981,180.

In some embodiments, the identifier is a color, such that particles of different sets have different colors. In some embodiments, the identifier is a different size, wherein particles of different sets have different sizes. In some embodiments, the identifier is both different colors and different sizes. Such means for distinguishing particles are known. For example, U.S. Pat. No. 4,499,052 (Fulwyler, “Apparatus for distinguishing multiple subpopulations of cells”) describes an encoded-particle multiplexed assay method utilizing beads as the particles, wherein the bead type is distinguished by color and/or size.

As used herein, by “binding agent” means any agent capable of forming a physical or chemical association with a glyco-molecule. Binding agents of the invention include, without limitation, antibodies, modified antibodies (including antibody fragments, such as Fab fragments), lectins, proteins, glycoproteins, and aptamers capable of binding glyco-molecules. In short, any compound or chemical capable of binding a glycol-molecule is a binding agent of the invention. All the particles coated with one type of binding agent (e.g., an antibody specific for the glycoprotein interleukin-8) are encoded with the same identifier-these identical particles make up a set of particles according to the invention.

In accordance with the invention, each binding agent is coated onto (i.e., immobilized on or affixed to) identically encoded particles. In some embodiments, where the binding agent is in solution, the particles can simply be soaked in the solution until the particles are coated with the binding agent. In another embodiment, the solution containing the binding agent can be used to “paint” the particles, and the binding agents allowed to dry onto the particles, thus coating the particles.

The invention thus provides sets of encoded particles which can be distinguished from other sets of encoded particles, and labeled molecules. If the molecule is glycosylated and is bound by a binding agent, the particle to which the binding agent is attached will be bound by the encoded particle. For example, there may be three different sets of particles, distinguishable in that their diameters are 5.0 μm, 5.5 μm, and 6.0 μm, coated with the following binding agents respectively: wheat germ agglutinin (WGA), a lectin that binds to glycosylated molecules containing the sequence GlcNAc beta1-4 Man beta1-4 GlcNAc beta1-4 GlcNAc-Asn; an antibody that specifically binds to Type II collagen; the Narcisss pseudonarcissus agglutinin (NPA), a lectin that bind to glycosylated molecules containing alpha-1,3 mannobiose. A sample labeled with the Texas Red dye may be added to a mixture containing particles from all three sets. By measuring the particles individually with a reading instrument (e.g., a cytometer), the size particle which is bound to the Texas Red labeled molecule is readily determined.

The invention also covers uncovering carbohydrate residues on a glycosylated molecule by treatment with glycosidases, followed by re-analysis on the encoded particles. Thus, a glycosylated molecule in a sample may have an unknown glycosylation pattern, and the invention a means for characterizing the structure of the carbohydrate residues on the glycosylated molecule. For example, a FITC labeled sample may be contacted with a plurality of particle sets, and the data collected for those particles that have bound FITC. In some embodiments, the FITC is used to label the reducing end of the carbohydrates in the sample. Once that data is collected, the sample (still contacting the plurality of particle sets) is treated with a glycosidase, and then either eluted from the old particles and allowed to contact a fresh set, or simply allowed to recontact particles in the original set. Glycosidase treatment will often reveal masked carbohydrate residues that were previously unavailable for binding to a binding agent-coated particle due to another carbohydrate residue. In this way, further characterization of a glycosylated molecule in the sample may be accomplished.

Thus, in a further aspect, the invention provides a method for characterizing a sugar residue on a glycosylated molecule, comprising contacting the glycosylated molecule with a plurality of particle sets, wherein each particle in a particle set is coated with a glycosylated molecule specific binding agent and wherein each particle in the particle set is encoded with a different identifier and collecting identifier data and collecting binding agent interaction data. The glycosylated molecule is next treated with a glycosidase, and then allowed to contact the plurality of particle sets. Identifier data and binding agent data are collected, where the identifier data and the binding agent interaction data characterize one or more sugar residues on the glycosylated molecule. In some embodiments, the reducing end of a carbohydrate residue on the glycosylated molecule is labeled.

The invention also provides a kit for performing simultaneous assays of one or more glycosylated molecules in a sample. The kit includes one or more aliquots comprising a plurality of particle sets, wherein each particle in a particle set is coated with a glycosylated molecule specific binding agent and wherein each particle in the particle set is encoded with a different identifier, and a reagent for attaching a label to the glycosylated molecule.

Additionally, for characterization of glycosylated molecules, the invention provides a kit for characterizing a carbohydrate residue on a glycosylated molecule. The kit includes one or more aliquots comprising a plurality of particle sets, wherein each particle in a particle set is coated with a glycosylated molecule specific binding agent and wherein each particle in the particle set is encoded with a different identifier; a glycosidase; and a reagent for attaching a label to the glycosylated molecule. In some embodiments, reagent attaches the label to the reducing end of a carbohydrate residue on the glycosylated molecule.

The kits of the invention may also include instructions for using the kit.

In some embodiments, the kits further include a vessel in which to perform the assay. The vessel, in accordance with the invention, can be anything in which a sample can be contacted with a plurality of particle sets. Thus, non-limiting examples of a vessel include the well of a microtiter plate, a scintillation vial, a test tube, a tissue culture plate, a beaker, a vial, a microfuge tube, and the like.

The invention further provides a system for performing simultaneous assays of one or more glycosylated molecules in a sample. The system includes one or more aliquots comprising a plurality of particle sets, wherein each particle in a particle set is coated with a glycosylated molecule specific binding agent and wherein each particle in the particle set is encoded with a different identifier, a vessel in which to perform the assay, a reagent for attaching a label to a glycosylated molecule, and a reading instrument.

In addition, the invention provides a system for characterizing a carbohydrate residue on a glycosylated molecule. The system includes one or more aliquots comprising a plurality of particle sets, wherein each particle in a particle set is coated with a glycosylated molecule specific binding agent and wherein each particle in the particle set is encoded with a different identifier; a glycosidase; a vessel in which to perform the assay; a reagent for attaching a label to a glycosylated molecule; and a reading instrument. In some embodiments, reagent attaches the label to the reducing end of a carbohydrate residue on the glycosylated molecule.

The kits and systems of the invention include a reagent for attaching labels to the glycosylated molecules. As mentioned above, any suitable label that is detectable with a reading machine may be used.

In accordance with the invention, any number of samples may be assayed. Thus, the number of different samples and the number of aliquots containing the plurality of particle sets can be any number. In some embodiments, the number of aliquots containing the plurality of particle sets is greater than the number of samples to be tested, so that positive and negative controls can be performed.

In particular embodiments, the glycosylated molecule specific binding agent is an antibody, a lectin, and a glycoprotein.

In some embodiments, a plurality of multiplex assays is constructed by pooling several sets of identically coated and encoded particles, thus forming a mixture of encoded particles, each particle type coated with a unique and identifiable specific binding agent. In some embodiments, the binding agent is labeled with a fluorescent dye, such as cyanine 3, that has a detectable excitation and emission wavelengths. For example, where the binding agent is an antibody that specifically binds to a particular glycosylated molecule (e.g., type I collagen) is labeled with a fluorescent dye or fluorophore (e.g., cyanine 3) prior to its use in the invention. Thus, in some embodiments, the identifier data and binding agent interaction data are collected by reading instruments. In some embodiments, the identifier data and the binding agent interaction data are collected by the same reading instrument. For example, where the particle is labeled with a fluorescent label, the same instrument can be employed to collect the optical signals from both the particle and from the binding agent.

From the pool of several sets of particles are formed aliquots of approximately equal numbers of each type of particles. In some embodiments, where the particles are in suspension, the particles are handled by pipetting after agitation drives them into suspension. In other words, no nanoliter-scale printing is required. The multiplexed assay is also typically incubated in a microplate well, a vial, or a tube, again with simple liquid transfer by pipette.

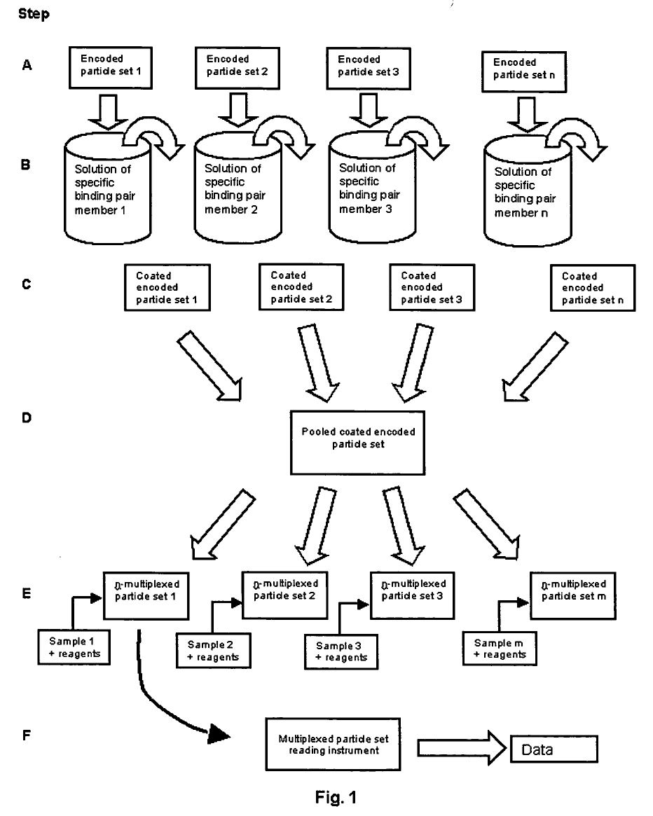

Referring to FIG. 1, the general Steps A through F are followed in preparing and performing an encoded particle multiplex assay in accordance with the invention. In the non-limiting example shown in FIG. 1, the number of analytes, namely specific glycoproteins in this non-limiting example, to be assayed is defined as n. In Step A of FIG. 1, sets of encoded particles 1 through “n”, wherein each particle in each set is encoded with the same identification code, are provided and kept separate. In Step B, each set of encoded particles is reacted with a different solution containing a binding agent that will specifically bind to the analyte to be assayed for, such as a glycoprotein-specific antibody, a glycoprotein, or a lectin. Thus, each set of particles contains particles that are encoded with identical barcodes and are coated with identical binding agents.

In Step C of FIG. 1, the encoded, coated particles have been removed from the coating solution. This can be done by pipetting, or by filtering, centrifugation, or any other standard laboratory process for separating solid particles from liquids. The result at this Step C is the production of “n” separate sets of coated, encoded particles wherein all of the beads in each set has the same code and coating, but the codes and coatings differ between the sets. In some embodiments, “n” is a number between about 2 and about 20,000. In some embodiments, “n” is a number between about 2 and about 15,000. In some embodiments, “n” is a number between about 2 and about 10,000. In some embodiments, “n” is a number between about 2 and about 5,000. In some embodiments, “n” is a number between about 2 and about 2,000. In some embodiments, “n” is a number between about 2 and about 1,000. In some embodiments, the “n” is a number between about 2 and about 200. In some embodiments, the “n” is a number between about 5 and about 100. In some embodiments, the “n” is a number between about 5 and about 50.

In Step D of FIG. 1, these “n” sets are pooled together and mixed, for example, in an aqueous buffer. At Step E of FIG. 1, aliquots of particles have been taken from the pooled set to form a plurality (“m”) of “n”-multiplexed particle sets. The “m” number may be any number, depending upon how many samples are being assayed. As mentioned above, the number “m” may be larger than the actual number of samples to be tested, to allow for positive and negative controls. Thus, the number “m” may be any number, such as a number between about 2 and about 20,000. In some embodiments, “m” is a number between about 2 and about 15,000. In some embodiments, “m” is a number between about 2 and about 10,000. In some embodiments, “m” is a number between about 2 and about 5,000. In some embodiments, “m” is a number between about 2 and about 2,000. In some embodiments, “m” is a number between about 2 and about 1,000. In some embodiments, “m” is a number between about 2 and about 150.

Each of these “m” aliquots at Step E in FIG. 1 typically has a nominally identical number of particles in it and nominally equal populations of each of the n species of particles as compared to the other “m” aliquots. The distributions deviate from the nominal conditions due to randomness and tolerances on the mixing of particles within the pool and the aliquotting process. Typically, the aliquots are sized so that they contain multiple replicates of each bead type, anywhere from about 3 to about 5,000 for example. In other words, each “m” aliquots contains more than one set, wherein each set contains from about 1 to about 5,000 member particles. By aliquoting replicates beyond the minimum number of particles needed to generate a valid signal, the user can be assured that all analytes will be assayed for with each n-multiplexed particle set.

The aliquots at Step E of FIG. 1 are each utilized to perform an n-multiplexed assay on a plurality of samples, where the plurality is less than or equal to “m”. In other words, if m is 20, then 20 or fewer samples can be analyzed. In FIG. 1, the number of samples to be assayed by the collection of identical particle sets shown here is m.

In some non-limiting embodiments, each multiplexed assay is performed in a fluid-containing vessel such as a microplate well. Such a vessel would be loaded in the proper sequence with an aliquot containing “n” particle sets (i.e., an aliquot containing “n” different sets), a sample to be assayed, and with the other reagents such as labeling and washing reagents. After one or more incubation periods, the particle set with the labeled assayed analytes bound to each particle is removed from the assay vessel and transferred to a reading instrument at Step F of FIG. 1. The reading instrument (e.g., a computer) reads the identifier encoded onto the particle and the associated one or more label signals from the binding agent coating each particle. Signals from replicate particles with the same codes and coatings may be consolidated in the instrument or in a downstream data processing computer.

The encoded particle multiplex assay process shown in FIG. 1 can be applied to a variety of specific binding assays. The invention thus provides a system, kit, and methods wherein the barcode-encoded particles are coated with glycosylated molecule-specific binding agents, and the described multiplex assay detects and measures a plurality of glycosylated molecules in each sample.

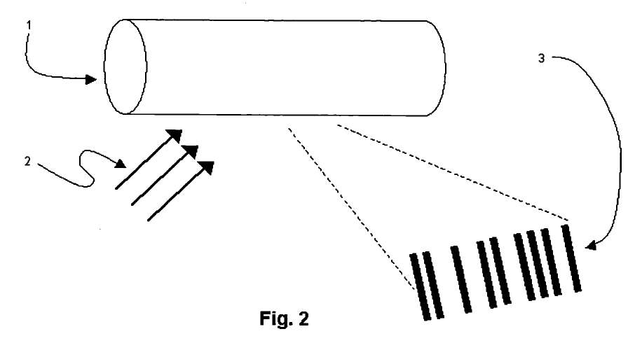

FIG. 2 depicts an encoded particle that utilizes a hologram or diffraction grating recorded inside the particle to record a barcode or other identifier. The particle 1 is interrogated by a beam of parallel light 2 at a controlled wavelength and incidence angle. For example, the beam may be a laser beam and the particle may be cylindrical and oriented to the beam in a transparent flow capillary or by lying in an oriented groove in a grooved particle-reading plate. Such a cylindrical particle can, for example, be made from a length of glass fiber, with a diameter between about 10 μm and 100 μm and a length between about 25 μm and 250 μm. The holographic image 3, shown here as a barcode, is projected out from the particle at an orientation and image divergence set by the hologram recording conditions. In a preferred embodiment, the hologram image diverges as it projects away from the particle such that it is several mm long at a distance of about 10 to about 100 mm away from the particle. This allows the barcode to be read easily by a simple, inexpensive low-resolution imaging array such as a charge coupled device (CCD).

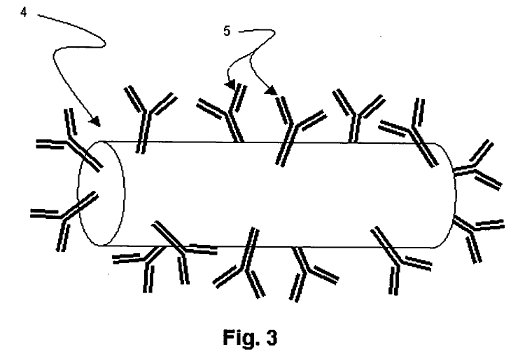

FIG. 3 depicts the cylindrical encoded particle 4 of FIG. 2 with antibodies 5 attached to its surface, for example, from the coating process described in FIG. 1 Steps B and C. In a particular embodiment for assaying glycosylated proteins, the antibodies are specific to glycosylation sites on specific proteins. The invention provides encoded particles coated with glycosylated molecule specific binding agents immobilized thereupon. The invention also provides kits containing one or more n-multiplexed sets of these coated, encoded particles (see FIG. 1), wherein each of sets includes at least one of coated, encoded particles coated with the specific binding agent that specifically binds to each of the intended analytes of a multiplexed glycosylated molecule assay.

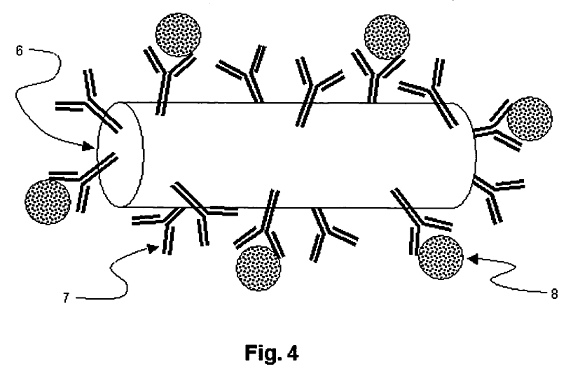

FIG. 4 depicts the coated, encoded particle 6 from FIG. 3 with some of the coated glycosylated molecule specific antibodies 7 bound to their complementary glycoproteins 8 after contact with a sample. In order to provide a signal to an encoded particle multiplex assay reading instrument, the glycosylated molecule must be labeled with a detectable molecule such as a fluorophore, chromophore, quantum dot or the like. Such labeling may be applied to the glycosylated molecules prior to the encoded particle multiplex assay or after. Further, the labeling may be of the “sandwich” variety wherein the label is conjugated to a secondary glycoprotein antibody to enhance specificity, or all of the glycoproteins or even all of the proteins in the sample may be labeled chemically or enzymatically. Thus, the invention provides methods and kits for such labeling of these glycosylated molecules prior to or after contact with the encoded, coated particles of the multiplexed assays.

The following examples illustrate the preferred modes of making and practicing the present invention, but are not meant to limit the scope of the invention since alternative methods may be utilized to obtain similar results.

### EXAMPLE 1

### Ratiometric Analysis of Two Glycoprotein Classes Present in Two Different Specimens

Certain lectins exhibit a high affinity for N-linked high mannose type, and hybrid type, as well as mono-antennary and bi-antennary complex type glycan structures. One of these lectins is concanavalin A (conA). Other lectins, such as wheat germ agglutinin (WGA), preferentially bind very tightly to the sequence GlcNAc beta1-4 Man beta1-4 GlcNAc beta1-4 GlcNAc -Asn.

The two lectins selected for this study are concanavalin A (conA) and wheat germ agglutinin (WGA). In the study, two well characterized model glycoproteins are evaluated to establish that the detection selectivities of conA and WGA, as defined by the multiplexed particle technology of the invention, are similar to those reported with high performance affinity chromatography and conventional lectin blotting. The outlined experiment demonstrates the ability of the encoded particles of the invention to be used as a substitute for affinity chromatography and lectin blotting and also demonstrates the ability to perform differential display glycoproteomics on the beads.

TABLE I
Lectin-based detection of model glycoproteins:

| | Percentage | Carbohydrate | Detection | Detection |
|---|---|---|---|---|
| Glycoprotein | carbohydrate | structure | with Con A | with WGA |
| Fetuin | 22% | Three N-linked | Weak | Strong |
| | | glycans of | | |
| | | tri-antennary | | |
| | | structure as | | |
| | | well as three | | |
| | | O-linked | | |
| | | glycans. | | |
| Horseradish | 22% | Eight N-linked | Strong | Weak |
| peroxidase | | glycans of | | |
| | | bi-antennary | | |
| | | structure. | | |

Two sets of particles, where a particle is identified as a member of a particle set by being encoding with a particular digital holographic code, are coated with either of two different lectins, concanavalin A (conA) or wheat germ agglutinin (WGA). The particles coated with conA have a binary code of 1001010100100111101000101, referred to as code A. The particles coated with WGA have a binary code of 1001010100100111101000000, referred to as code B. Both particle sets are then mixed together. Two binary mixtures of two glycoproteins, namely horseradish peroxidase (which is Con A positive) and fetuin (which is WGA positive), are prepared at ratios of 80:20 and 50:50, respectively. The first binary mixture (i.e., horseradish peroxidase) is labeled with Cy3-succinimidyl ester while the second (i.e., fetuin) is labeled with Cy5 succinimidyl ester. The two protein mixtures are combined and then are incubated with the encoded, coated particles. Unbound material is washed away using phosphate-buffered saline, pH 7.4 and then the Cy3 and Cy5 signals associated with each encoded particle is read to determine the abundances of the analytes. Addition of various nonionic detergents, surfactants, proteins and salts may be useful to reduce nonspecific binding in the experiment.

The results will show that the ratios of the two glycoproteins are accurately determined by this approach for both samples. Thus, from the mixture labeled with Cy3, approximately 80% of the Cy3 signal is associated with the code A particles, while only 20% of the signal will be found to be pulled down by the code B particles. Similarly, from the mixture labeled with Cy5, 50% of the Cy5 label will be found to be pulled down by the code A particles and approximately 50% by the code B particles.

This simple example can be extended to the analysis of various glycoprotein classes in clinical specimens. The metastatic spread of tumor cells in malignant progression is known to be a major cause of cancer mortality. Protein glycosylation is increasingly being recognized as one of the most prominent biochemical alterations associated with malignant transformation and tumorigenesis. The multiplexed assay of the invention will allow the parallel determination of altered glycosylation patterns within a single experiment. In certain disease states, the relative abundances and branching structures of glycans are often altered relative to the normal state, and these alterations in glycosylation may be indicative of the stage of the disease and thus useful for diagnosis.

### EXAMPLE 2

### Structural Analysis of Oligosaccharides

Lectin affinity chromatography has been used previously for the structural analysis of carbohydrates. One major drawback related to this method is the need to use an increasing number of lectin affinity columns to reach highly accurate structural determination, which considerably slows down the analytical procedure and consumes significant amounts of analyte. What is needed is a multiplexed approach wherein lectins are combined in a single reaction mixture and binding selectivities are then read out in parallel. Such a method is described in this example.

The structures of asparagine-linked oligosaccharides (N-linked) fall into three main categories, namely. high mannose, hybrid, and complex type. They all share the common core structure, Man alpha1-3(Man alpha1-6)Man beta1-4GlcNAc beta1-4GlcNAc-Asn, but differ in their outer branches. The complex type structures may be modified both by addition of extra branches on the alpha-mannose residues or by addition of extra sugar residues that elongate the outer chains or the core structures. Lectins having the core structure as essential specificity determinant are generally used first in the structural characterization of oligosaccharides as they are able to discriminate the N- or O-linked (Ser/Thr-linked oligosaccharide) nature of the oligosaccharide-peptide linkage. The N-linked core structure is recognized by numerous lectins, but perhaps the most useful one is concanavalin A (conA). Wheat germ agglutinin (WGA) is another commonly used lectin that binds very tightly the glycopeptides containing the sequence GlcNAc beta1-4 Man beta1-4 GlcNAc beta1-4 GlcNAc -Asn. The presence of an alpha1-6 fucose residue on the N-Acetyl glucosamine residue linked to the asparagine (“core” fucosylation) may be specifically identified by the use of Aleuria aurentia lectin. Sambucus nigra agglutinin (SNA) lectin is used to selectively assay the concentration of sialic acid containing glycopeptides.

In this example, the four lectins listed above (namely conA, WGA, Aleuria aurentia lectin, and SNA lectin, are affixed to (i.e., used to coat) four different encoded particle sets by standard methods. Bovine serum albumin or triethanolamine is conjugated to a fifth particle set and simply serves as a negative control in the experiment. Other lectins with different selectivities could be employed to obtain additional information about carbohydrate structure. In this example, however, the goal is to determine whether an oligosaccharide is N-linked or O-linked, whether it is sialyated and whether it is fucosylated.

Single oligosaccharide chains with few amino-acids are generally a good starting material for carbohydrate characterization by the described method, and are easily prepared using proteolytic enzymes. Carbohydrates may be prepared from a variety of test glycoproteins or from clinically relevant material, such as serum. Lectin-carbohydrate interactions are relatively weak (K_{d}=10⁻⁴ to 10⁻⁷ M), compared with antigen-antibody interactions (K_{d}=10⁻⁸ to 10⁻¹² M). Typically, carbohydrate concentrations of 100 nM are sufficient for the outlined assay. Lactoferrin, an 80 kDa iron-binding glycoprotein found mainly in milk and in polymorphonuclear leukocytes, is used as the model glycoprotein in this example. The glycoprotein consists of a 689 amino acid polypeptide chain to which complex and high-mannose-type carbohydrates are linked. Lactoferrin is isolated from a pool of bovine colostrum by carboxymethyl cation exchange chromatography and then is cleaved with cyanogen bromide and V8 protease. Carbohydrates are then released from glycopeptides by gas-phase hydrazinolysis (100° C., 2 hours) with a Hydraclub C-206 instrument (Honen Co., Tokyo, Japan). The resulting carbohydrates are subsequently labeled by reaction at their reducing termini with any of a variety of fluorescent carbohydrate-labeling reagents, such as anthranilic acid (2-aminobenzoic acid; 2-AA), 1-phenyl-3-methyl-5-pyrazolone (PMP), phenylhydrazine (PHN), 3-acetylamino-6-aminoacridine or 2-aminopyridine.

In this example, the carbohydrates are N-acetylated and derivatized with PMP. The resulting carbohydrate derivatives are readily detectable based upon their fluoresce properties, with 480 nm for excitation and 530 nm for emission maxima. The modified carbohydrates are purified by HPLC on a column of PALPAK Type-S, as follows. After the labeling reagents and byproducts are eluted with 500 mM acetic acid-triethylamine (pH 7.3)/acetonitrile/water (10:75:15, vol %), the modified carbohydrates are eluted with 500 mM acetic acid-trimethylamine (pH 7.3)/acetonitrile/water, (!0:50:40, vol %). This material is subsequently dried down using a Savant SpeedVac evaporator, resuspended in phosphate-buffered saline and employed in the multiplexed particle assay of the invention.

Labeled material is incubated with encoded particle sets that have the salient lectins affixed to their surfaces. Typically, incubation is performed at room temperature for 1 hour, on a plate shaker with shaking at 550 rpm. After extensive washing with phosphate-buffered saline, pH 7.4, co-localization of the fluorescent carbohydrate and the encoded particle is used to establish structural features of the carbohydrate. A Luminex 100 cytometer is used to read the particles. The Luminex 100 cytometer contains two solid state lasers: a reporter laser (532 nm, nominal output 12.0-16.5 mW that excites fluorescent molecule (e.g., Cy3 or Cy5) on the glycosylated molecule bound to the particles, and a classification laser (635 nm, nominal output 8.6-9.6 mW) that excites the fluorochrome coated within the particle. The reporter emission spectrum does not overlap with the classification emission signal. When the particles are excited at a wavelength of 532 nm, Cy3 emits light at 570 nm. The extent of non-specific binding can be measured using bovine serum albumin or triethanolamine-modified particle probes. The level of non-specific binding depends on the glycoprotein, so that the net binding of various glycoproteins to the lectin-coated particles can be calculated by comparing the mean fluorescence intensity of the bound Cy3 dye to that obtained using the triethylanolamine-modified particle probes.

The labeled glycosylated molecule associates with three bead sets (concanavalin A, Aleuria aurentia lectin and Sambucus nigra agglutinin lectin) indicating a sialylated, fucosylated, N-linked carbohydrate. Lactoferrin is known to contain N-glycosidically-linked carbohydrates possessing N-acetylneuraminic acid, galactose, mannose, fucose, N-acetylglucosamine, and N-acetylgalactosamine, and thus the results are consistent with expectations. Little or no association of the carbohydrate is detected on the bovine serum albumin-conjugated particles and buffer conditions may be adjusted by inclusion of various surfactants, detergents (e.g., 0.05% Tween-20), proteins (e.g., 1% bovine serum albumin), chaotropes or salts should some nonspecific binding be detectable.

Such analysis of a carbohydrate structure using a battery of particle-bound lectins with different selectivities not only helps establish the identity of a carbohydrate or suggests approaches to the purification of a carbohydrate to homogeneity from among a mixture of different carbohydrates, but also successfully assays the microheterogeneity in these carbohydrates, which is an otherwise impracticable problem to address. For example, with the labeled carbohydrate described in the example, partial core alpha1-6 fucosylation results in distribution of the carbohydrate between the Aleuria aurentia lectin-labeled encoded particles and the wheat germ agglutinin-labeled encoded particles. The reason for this distribution becomes apparent as unmasking of lectin-binding sites is discussed. The basic structural approach outlined in this example can be further refined by employing complementary approaches that involve chemical and/or enzymatic treatment (using glycoenzymes, glycosidases and glycosyltransferases) to unmask target carbohydrate sites. For example, after association of carbohydrates with the different particle sets is determined, the glycoproteins are incubated with a particular glycosidase. Treatment with the glycosidase may reveal a new carbohydrate site which, in turn, can bind to one of the lectin-coated encoded particles. The treated glycoprotein is then analyzed for redistribution of the labeled carbohydrates. In this manner, masked lectin-binding sites may be revealed after removal of certain capping carbohydrates. For example, the interaction of wheat germ agglutinin (WGA) is only possible when the alpha1-6 fucose present in the native structure is missing. Treatment of a fucosylated carbohydrate with fucosidase can lead to redistribution of carbohydrates from the Aleuria aurentia lectin particles to the wheat germ agglutinin (WGA) particles. In other words, although the glycoprotein originally did not bind to the WGA coated particles, but did bind to the Aleuria aurentia lectin particles, following treatment with fucosidase, the treated carbohydrate can now bind to the WGA coated particles.

The results described in this example indicate that the multiplex particle approach of the invention is sensitive to changes in the content of carbohydrate residues known to be present in serum glycoproteins, and has the potential to be used to screen serum proteins for glycosylation changes due to disease. In addition, the use of glycosidases to induce specific structural changes in glycoproteins can support the development of particle-based formats specific for detecting changes in the glycoproteome of certain diagnostic fluids and types of disease. While the example provided uses a limited set of four lectins, large batteries of lectins having differing and sometimes overlapping selectivities may be used in this same basic approach. In order to exploit the full potential of this particle-based approach, integrated lectin recognition and proprietary algorithms, database and software to obtain quantitative data on the structure, sequence and proportion of carbohydrates in a glycoprotein sample is required. Databases of experimentally determined K_{d} values for thousands of individual lectin-oligosaccharide interactions, facilitate interpretation of such encoded particle experiments.

### EXAMPLE 3

### Real-time Analysis

Sensitive, real-time observation of multiple lectin-carbohydrate interactions under equilibrium conditions permit measurements to be performed without intervening washing steps. This is particularly advantageous when lectins are employed as the binding agents, due to the relatively weak K_{D} of lectin-glycan interactions relative to lectin-antibody interactions. As such, detection of glycan binding to encoded particles may be achieved by scintillation proximity assay (SPA), as described in Patton, W. U.S. Patent Application Ser. No. 60/707,492 (Aug. 11, 2005, “Buoyancy-compensated beads suitable for proximity assays”). Briefly, a scintillating phosphor is deposited on the surface of coding microscopic beads or particles, each of which is used for measuring a different assay component. For example, particles may be dyed with differing concentrations of two fluorophores to generate distinct particles sets, as is performed with Luminex beads. Each particle set is coated with a layer of inorganic phosphor and then a capture lectin specific for one particular analyte. The analyte is labeled with a radioisotope such as ³H, ¹²⁵I, ¹⁴C, ³⁵S or ³³P, that emits low energy radiation, which is easily dissipated in an aqueous-based environment. The amount of captured analyte is subsequently detected based upon the magnitude of the scintillation signal of the inorganic phosphor coating, which is in direct proportion to the amount of analyte bound. The identity of the analyte is determined from the characteristic fluorescence properties of the core bead itself, as determined based upon color ratios. While described with specific reference to the SPA, luminescence-based proximity assays (fluorescent, phosphorescent, chemiluminescent), such as homogenous time-resolved fluorescence and fluorescence polarization assays, may also be practiced using the cited method. In the case of homogenous time-resolved fluorescence assays, the energy donor is the rare earth dopant in the inorganic phosphor coating, rather than radioactivity. The energy acceptor is a fluorophore affixed to the glycan whose excitation profile overlaps the emission profile of the dopant in the inorganic phosphor. Binding events are detected as emission of the longer wavelength fluorophore upon excitation of the shorter wavelength emitting phosphor with mid-range ultraviolet radiation.

Although specific embodiments have been illustrated and described herein, it will be appreciated by those skilled in the art that any arrangement which is calculated to achieve the same purpose maybe substituted for the specific embodiments shown. This application is intended to cover any adaptations or variations of the present invention.

1. A method for simultaneously detecting one or more glycosylated molecules in a number of labeled samples, comprising
contacting the number of labeled samples with a number of aliquots comprising a plurality of particle sets, wherein each particle in a particle set is coated with a glycosylated molecule specific binding agent and wherein each particle in the particle set is encoded with a different identifier, and wherein the number of samples is greater than or equal to the number of aliquots containing the plurality of particle sets; and
collecting identifier data and collecting binding agent interaction data, wherein the identifier data and the binding agent interaction data indicate the presence of one or more glycosylated molecules in the number of samples.

2. The method of claim 1, wherein the glycosylated molecule is selected from the group consisting of a glycoprotein, proteoglycan, oligosaccharide, lipopolysaccharide, glycopeptide, glycosaminoglycan, polysaccharide, glycolipid, ganglioside, glycohormone, cerebroside, and glycosylsphingolipid.

3. The method of claim 1, wherein the glycosylated molecule specific binding agent is selected from the group consisting of an antibody, a lectin, an aptamer, a protein, and a glycoprotein.

4. The method of claim 1, wherein the identifier data and binding agent interaction data are collected by a reading instrument.

5. The method of claim 1, wherein the sample is a biological sample.

6. The method of claim 1, wherein the binding agent interaction data is collected by fluorescence detection.

7. The method of claim 1, wherein the identifier is a barcode or a fluorescent label.

8. The method of claim 1, wherein the plurality of particle sets is between about 2 and about 400.

9. The method of claim 1, wherein each particle set comprises between about 1 and about 5,000 particles.

10. A method for characterizing a carbohydrate residue on a labeled glycosylated molecule, comprising:
(a) contacting the labeled glycosylated molecule with a plurality of particle sets, wherein each particle in a particle set is coated with a glycosylated molecule specific binding agent and wherein each particle in the particle set is encoded with a different identifier;
(b) collecting identifier data and collecting binding agent interaction data;
(c) treating the product of step (b) with a glycosidase; and
(e) repeating steps (a) and (b) at least once, wherein the identifier data and the binding agent interaction data characterize one or more carbohydrate residues on the glycosylated molecule.

11. The method of claim 10, wherein the glycosylated molecule is selected from the group consisting of a glycoprotein, proteoglycan, oligosaccharide, lipopolysaccharide, glycopeptide, glycosaminoglycan, polysaccharide, glycolipid, ganglioside, glycohormone, cerebroside, and glycosylsphingolipid.

12. The method of claim 10, wherein the glycosylated molecule specific binding agent is selected from the group consisting of an antibody, a lectin, an aptamer, a protein, and a glycoprotein.

13. The method of claim 10, wherein the identifier data and binding agent interaction data are collected by a reading instrument.

14. The method of claim 10, wherein the binding agent interaction data is collected by fluorescence detection.

15. The method of claim 10, wherein the identifier is a barcode or a fluorescent label.

16. The method of claim 10, wherein the plurality of particle sets is between about 2 and about 400.

17. The method of claim 10, wherein each particle set comprises between about 1 and about 5,000 particles.

18. A kit for performing simultaneously assaying a one or more glycosylated molecules in a sample, comprising:
one or more aliquots comprising a plurality of particle sets, wherein each particle in a particle set is coated with a glycosylated molecule specific binding agent and wherein each particle in the particle set is encoded with a different identifier, and
a reagent for attaching a label to a glycosylated molecule.

19. The kit of claim 18, wherein the plurality of particle sets in the kit is between about 2 and about 400.

20. The kit of claim 18, wherein the identifier is a barcode or a fluorescent label.

21. The kit of claim 18, wherein the glycosylated molecule specific binding agent is selected from the group consisting of an antibody, a lectin, an aptamer, a protein, and a glycoprotein.

22. The kit of claim 18, further comprising a vessel in which to perform the assay.

23. The kit of claim 18, further comprising instructions for using the kit to perform the assay.

24. A kit for characterizing a carbohydrate residue on a glycosylated molecule, comprising one or more aliquots comprising a plurality of particle sets, wherein each particle in a particle set is coated with a glycosylated molecule specific binding agent and wherein each particle in the particle set is encoded with a different identifier; a glycosidase; and a reagent for attaching a label to the glycosylated molecule.

25. The kit of claim 24, wherein the plurality of particle sets in the kit is between about 2 and about 400.

26. The kit of claim 24, wherein the identifier is a barcode or a fluorescent label.

27. The kit of claim 24, wherein the glycosylated molecule specific binding agent is selected from the group consisting of an antibody, a lectin, an aptamer, a protein, and a glycoprotein.

28. The kit of claim 24, further comprising a vessel in which to perform the assay.

29. The kit of claim 24, further comprising instructions for using the kit to perform the assay.

30. A system for simultaneously assaying a one or more glycosylated molecules in a sample, comprising:
one or more aliquots comprising a plurality of particle sets, wherein each particle in a particle set is coated with a glycosylated molecule specific binding agent and wherein each particle in the particle set is encoded with a different identifier;
a vessel in which to perform the assay;
a reagent for attaching a label to a glycosylated molecule; and
a reading instrument.

31. The system of claim 30, wherein the plurality of particle sets in the kit is between about 2 and about 400.

32. The system of claim 30, wherein the glycosylated molecule specific binding agent is selected from the group consisting of an antibody, a lectin, an aptamer, a protein, and a glycoprotein.

33. The system of claim 30, wherein the identifier is a barcode or a fluorescent label.

34. A system for characterizing a carbohydrate residue on a glycosylated molecule, comprising:
one or more aliquots comprising a plurality of particle sets, wherein each particle in a particle set is coated with a glycosylated molecule specific binding agent and wherein each particle in the particle set is encoded with a different identifier;
a glycosidase
a vessel in which to perform the assay;
a reagent for attaching a label to a glycosylated molecule; and
a reading instrument.

35. The system of claim 34, wherein the plurality of particle sets in the kit is between about 2 and about 400.

36. The system of claim 34, wherein the glycosylated molecule specific binding agent is selected from the group consisting of an antibody, a lectin, an aptamer, a protein, and a glycoprotein.

37. The system of claim 34, wherein the identifier is a barcode or a fluorescent label.
